(19) United States
(12) Patent Application Publication
(10) Pub. No.: US 2008/0140070 A1
(43) Pub. Date: Jun. 12, 2008

(54) MULTI-ELECTRODE APPARATUS FOR TISSUE WELDING AND ABLATION

(75) Inventors: DOMINIQUE J. FILLOUX, Redwood City, CA (US); MIRIAM H. TAIMISTO, San Jose, CA (US); UDAY N. KUMAR, San Francisco, CA (US); VENKATA VEGESNA, Sunnyvale, CA (US)

Correspondence Address: CIERRA, INC. &;TOWNSEND & TOWNSEND AND CREW, LLP, TWO EMBARCADERO CENTER, EIGHTH FLOOR, SAN FRANCISCO, CA, 94111-3834, US

(73) Assignee: CIERRA, INC., Redwood City, CA (US)

(21) Appl. No.: 11/757,875

(22) Filed: Jun. 4, 2007

Related U.S. Application Data
(60) Provisional application No. 60/869,049, filed on Dec. 7, 2006.

(51) Int. Cl. A61B 18/18

(52) U.S. Cl. 606/41

(57) ABSTRACT

An apparatus for delivering energy to tissue, comprising: an elongate flexible shaft having a proximal end and a distal end; a sheath disposed over at least a portion of the flexible shaft; a housing provided on the distal end of the flexible shaft; a plurality of electrodes mounted on the housing, the electrodes having a tissue apposition surface having a non-coplanar shape that conforms to the anatomy of a patient.

### CROSS-REFERENCES TO RELATED APPLICATIONS

This application is a non-provisional of U.S. Provisional Application No. 60/869,049 (Attorney Docket No. 022128-001600US), the entire contents of which are incorporated herein by reference.

### BACKGROUND OF THE INVENTION

The invention generally relates to medical devices and methods. More specifically, the invention relates to energy based devices, systems and methods for treatment of patent foramen ovale.

Fetal blood circulation is much different than adult circulation. Because fetal blood is oxygenated by the placenta, rather than the fetal lungs, blood is generally shunted away from the lungs to the peripheral tissues through a number of vessels and foramens that remain patent (i.e., open) during fetal life and typically close shortly after birth. For example, fetal blood passes directly from the right atrium through the foramen ovale into the left atrium, and a portion of blood circulating through the pulmonary artery trunk passes through the ductus arteriosis to the aorta.

At birth, as a newborn begins breathing, blood pressure in the left atrium rises above the pressure in the right atrium. In most newborns, a flap of tissue closes the foramen ovale and heals together. In approximately 20,000 babies born each year in the US, the flap of tissue is missing, and the hole remains open as an atrial septal defect (ASD). In a much more significant percentage of the population (estimates range from 5% to 20% of the entire population), the flap is present but does not heal together. This condition is known as a patent foramen ovale (PFO). Whenever the pressure in the right atrium rises above that in the left atrium, blood pressure can push this patent channel open, allowing blood to flow from the right atrium to the left atrium.

Patent foramen ovale has long been considered a relatively benign condition, since it typically has little effect on the body's circulation. More recently, however, it has been found that a significant number of strokes may be caused at least in part by PFO. In some cases, stroke may occur because a PFO allows blood containing small thrombi to flow directly from the venous circulation to the arterial circulation and into the brain, rather than flowing to the lungs where the thrombi can become trapped and gradually dissolved. In other cases, thrombi might form in the patent channel of the PFO itself and become dislodged when the pressures cause blood to flow from the right atrium to the left atrium. It has been estimated that patients with PFOs who have already had cryptogenic strokes have a 4% risk per year of having another stroke.

Further research is currently being conducted into the link between PFO and stroke. At the present time, if someone with a PFO has two or more strokes, the healthcare system in the U.S. may reimburse a surgical or other interventional procedure to definitively close the PFO. It is likely, however, that a more prophylacetic approach would be warranted to close PFOs to prevent the prospective occurrence of a stroke. The cost and potential side-effects and complications of such a procedure must be low, however, since the event rate due to PFOs is relatively low. In younger patients, for example, PFOs sometimes close by themselves over time without any adverse health effects.

Another highly prevalent and debilitating condition—chronic migraine headache—has also been linked with PFO. Although the exact link has not yet been explained, PFO closure has been shown to eliminate or significantly reduce migraine headaches in many patients. Again, prophylacetic PFO closure to treat chronic migraine headaches might be warranted if a relatively non-invasive procedure were available.

Currently available interventional therapies for PFO are generally fairly invasive and/or have potential drawbacks. One strategy is simply to close a PFO during open heart surgery for another purpose, such as heart valve surgery. This can typically be achieved via a simple procedure such as placing a stitch or two across the PFO with vascular suture. Performing open heart surgery purely to close an asymptomatic PFO or even a very small ASD, however, would be very hard to justify.

A number of interventional devices for closing PFOs percutaneously have also been proposed and developed. Most of these devices are the same as or similar to ASD closure devices. They are typically “clamshell” or “double umbrella” shaped devices which deploy an area of biocompatible metal mesh or fabric (ePTFE or Dacron, for example) on each side of the atrial septum, held together with a central axial element, to cover the PFO. This umbrella then heals into the atrial septum, with the healing response forming a uniform layer of tissue or “pannus” over the device. Such devices have been developed, for example, by companies such as Nitinol Medical Technologies, Inc. (Boston, Mass.) and AGA Medical, Inc. (White Bear Lake, Minn.). U.S. Pat. No. 6,401,720 describes a method and apparatus for thoracoscopic intracardiac procedures which may be used for treatment of PFO.

Although available devices may work well in some cases, they also face a number of challenges. Relatively frequent causes of complications include, for example, improper deployment, device embolization into the circulation and device breakage. In some instances, a deployed device does not heal into the septal wall completely, leaving an exposed tissue which may itself be a nidus for thrombus formation. Furthermore, currently available devices are generally complex and expensive to manufacture, making their use for prophylacetic treatment of PFO impractical. Additionally, currently available devices typically close a PFO by placing material on either side of the tunnel of the PFO, compressing and opening the tunnel acutely, until blood clots on the devices and causes flow to stop.

Research into methods and compositions for tissue welding has been underway for many years. Such developments are described, for example, by Kennedy et al. in “High-Burst Strength Feedback-Controlled Bipolar Vessel Sealing,” Surg. Endosc. (1998) 12:876-878. Of particular interest are technologies developed by McNally et. al., (as shown in U.S. Pat. No. 6,391,049) and Fusion Medical (as shown in U.S. Pat. Nos. 5,156,613, 5,669,934, 5,824,015 and 5,931,165). These technologies all disclose energy delivery to tissue solders and patches to join tissue and form anastamoses between arteries, bowel, nerves, etc. Also of interest are a number of patents by inventor Sinofsky, relating to laser suturing of biological materials (e.g., U.S. Pat. Nos. 5,725,522, 5,569,239, 5,540,677 and 5,071,417). None of these disclosures, however, show methods or apparatus suitable for positioning the tissues of the PFO for welding or for delivering the energy to a PFO to be welded.

Causing thermal trauma to a patent ovale has been described in two patent applications by Stambaugh et al. (PCT Publication Nos. WO 99/18870 and WO 99/18871). The devices and methods described, however, cause trauma to PFO tissues in hopes that scar tissue will eventually form and thus close the PFO. Using such devices and methods, the PFO actually remains patent immediately after the procedure and only closes sometime later (if it closes at all). Therefore, a physician may not know whether the treatment has worked until long after the treatment procedure has been performed. Frequently, scar tissue may fail to form or may form incompletely, resulting in a still patent PFO.

Therefore, it would be advantageous to have improved methods and apparatus for treating a PFO. Ideally, such methods and apparatus would help seal the PFO during, immediately after or soon after performing a treatment procedure. Also ideally, such devices and methods would leave no foreign material (or very little material) in a patient's heart. Furthermore, such methods and apparatus would preferably be relatively simple to manufacture and use, thus rendering prophylacetic treatment of PFO, such as for stroke prevention, a viable option. At least some of these objectives will be met by the present invention.

### BRIEF SUMMARY OF THE INVENTION

According to one aspect of the invention an apparatus for delivering energy to tissue is disclosed. The energy delivery apparatus includes an elongate flexible shaft having a proximal end and a distal end; a first electrode operably connected to the elongate flexible shaft; and a second electrode operably connected to the elongate flexible shaft and electrically independent from the first electrode, the second electrode at least partially surrounding and spaced apart from the first electrode. At least one of the first and second electrodes may include a non-planar tissue apposition surface. Moreover, the non-planar tissue apposition surface may be a continuous curve or a step.

The first electrode may be circular and the second electrode may be elongated. According to one variation, at least one of the first and second electrodes is generally rectangular. According to one variation, the second electrode may include a ring concentric with the first electrode. According to another one variation, at least one of the first and second electrodes includes at least two electrically coupled segments. The plurality of segments may be generally independently movable such that the segments generally conform to a patient's anatomy. The device may further include an intermediate electrode interposed between and spaced apart from the first and second electrodes. Moreover, the intermediate electrode may include at least two electrically coupled segments.

The aforementioned apparatus may further include a housing mounted to the distal end of the elongate flexible shaft, wherein the first and second electrodes are attached to the housing. Moreover, the housing may include at lease one area of diminished thickness configured to facilitate collapsing of the housing.

The aforementioned apparatus may further include a substrate mounted to the elongate flexible shaft, wherein the first and second electrodes are mounted on the substrate.

The aforementioned apparatus may further include at least one resistive bridge coupling the first and second electrodes, wherein the housing is adapted to be housed in a collapsed state within the sheath prior to deployment.

Also disclosed is an apparatus for delivering energy to tissue including an elongate flexible shaft having a proximal end and a distal end; a resilient housing mounted near the distal end of the flexible shaft; a first electrode mounted on the housing; a second electrode mounted on the housing; and a resistive bridge coupling the first and second electrodes.

Also disclosed is an apparatus for delivering energy to tissue, including an elongate flexible shaft having a proximal end and a distal end; a housing attached to the flexible shaft; at least three electrically independent electrodes operably connected to the housing. According to one aspect of the invention, the surface area of two of the electrodes is equal and differs from a surface area of the third three electrode. According to one aspect, at least one of the electrodes is generally rectangular.

Also disclosed is an apparatus for delivering energy to tissue, including an elongate flexible shaft having a proximal and distal end; a first electrode operably connected to the elongate flexible shaft; and at least one satellite electrode operably connected to the elongate flexible shaft, the satellite electrode(s) being electrically independent from the first electrode. According to one aspect, the at least one satellite electrode includes a plurality of satellite electrodes divided into at least two electrically independent groups. According to another aspect, the first group of satellite electrodes are disposed a first radial distance from the central electrode and the second group of satellite electrodes are disposed a second radial distance from the central electrode, the first distance radial being different from the second radial distance. The satellite electrodes may be disposed radially around the first electrode. According to one aspect, each of the satellite electrodes is equidistant from the first electrode. According to another aspect, the first electrode has a greater surface area than any given one of the plurality of satellite electrodes. Still further, the first electrode may include a plurality of electrically independent first electrodes adapted to be energized independently of one another.

An apparatus for delivering energy to tissue, including an elongate flexible shaft having a proximal end and a distal end; a sheath disposed over at least a portion of the flexible shaft; a housing provided on the distal end of the flexible shaft; a plurality of electrodes mounted on the housing, the electrodes having a tissue apposition surface having a non-coplanar shape that conforms to the anatomy of a patient. According to one aspect, the tissue apposition surface defines a continuous curve or a step.

An apparatus for delivering energy to tissue, including an elongate flexible member having a proximal end and a distal end, the distal end of the elongate member being predisposed to assume a first predefined shape; at least one electrode disposed on the elongate member proximate the distal end; and a sheath disposed over at least a portion of the elongate member and adapted to house the elongate member in an undeployed state in which the elongate member generally conforms to the shape of the sheath. According to one aspect, the at least one electrode may include at least one circumferential band disposed around the elongate member. According to another aspect of the invention, the predefined shape is generally one of an L-shape, a helix, a square, and a series of interlocking squares.

An apparatus for delivering energy to tissue, including: an elongate flexible member having proximal end and a distal end; an expandable and conformable member predisposed to assume a first predefined shape, the conformal member being operably connected to the distal end of the elongate member; and a plurality of electrodes disposed on the expandable member wherein at least some of the electrodes are electrically independent from the remaining electrodes, wherein the sheath is adapted to house the expandable member in a collapsed state prior to deployment. According to one aspect, the expandable member may include a balloon. Moreover, the balloon may include one of a continuously curved region and a stepped region.

An apparatus for delivering energy to tissue, including an elongate flexible member having a proximal end and a distal end; a sheath disposed over the elongate flexible member; a plurality of resilient members disposed attached to the elongate member and predisposed to assume a first predefined shape, wherein the resilient members are adapted to be housed in a collapsed state within the sheath prior to deployment; at least one energy delivery device formed on each the resilient member. According to one aspect, the self-expanding members are adapted to conform to a layered tissue defect. According to another aspect, the predefined shape is generally one of an L-shape, a spiral, a square shape, and a series of interlocking squares.

An apparatus for delivering energy to tissue, including an elongate flexible shaft having a proximal end and a distal end; a sheath disposed over at least a portion of the flexible shaft; a resilient housing near the distal end of the flexible shaft, the housing adapted to deflect so as to appose the tissue; and at least one electrode mounted to the distal housing; an elongated pusher coupled with one of the housing and the at least one electrode and adapted to deflect the at least one electrode into apposition with the tissue. According to one aspect, at least one of the housing and the electrode may include at lease one area of diminished thickness in which the housing/electrode is predisposed to collapse or deform.

An apparatus for delivering energy to tissue, including an elongate flexible shaft having a proximal end and a distal end; a sheath disposed over at least a portion of the flexible shaft; a resilient housing near the distal end of the flexible shaft, the housing adapted to deflect so as to appose the tissue; and at least one electrode mounted to the distal housing; a pusher coupled with and adapted to deflect the at least one of electrode into apposition with the tissue. According to one aspect, the distal housing may include at lease one area of diminished thickness in which the housing is predisposed to collapse or deform.

An apparatus for delivering energy to tissue, including an elongate flexible shaft having a proximal end and a distal end; a sheath disposed over at least a portion of the flexible shaft; a resilient substrate near the distal end of the flexible shaft; a plurality of compression members coupled with the substrate; and a plurality of electrodes spaced from one another and operably connected with the plurality of compression members, the plurality of electrodes adapted to individually advance or retract relative to the substrate so as to appose the tissue, wherein the substrate is adapted to be housed in a collapsed state within the sheath prior to deployment. According to one aspect, the plurality of compression members includes springs. According to another aspect, at least two of the plurality of electrodes is electrically isolated from one another.

An apparatus for delivering energy to tissue, including an elongate flexible shaft having a proximal end and a distal end; a sheath disposed over at least a portion of the flexible shaft; a plurality of electrically isolated electrodes, at least one the electrode being electrically insulated from another the electrode such that energy may be supplied to one electrode independent of the other electrodes; a resilient support structure operably connected to the shaft and movably supporting the plurality of electrodes such that each electrode is movable independent of others of the plurality of electrodes; wherein the resilient support structure is adapted to be housed in a collapsed state within the sheath prior to deployment. According to one aspect, the apparatus further includes a plurality of resilient members; one the resilient member interposed between the support structure and each the electrode. According to another aspect, the resilient members are electrically conductive and/or movably retain the electrodes within the resilient support structure. Moreover, the support structure may define a plurality of receptacles, with each the receptacle including a flange adapted to engage a corresponding lip formed on each electrode to retain the electrode within the receptacle.

A method for orienting an energy delivery device, including providing a catheter device having a plurality of electrically independent electrodes; guiding the catheter device to a target location using at least one of a guide wire and imaging means; measuring at least one of an impedance and electrocardiac conductivity between a given pair of electrodes and adjusting the orientation and or position of the catheter device in accordance with the measured value. The target location may be a PFO, and the imaging means may be one of TEE, TTE, and ultrasound. The measured value may be used to determine whether the electrode is biased posterior or anterior of one of the primum and secundum and/or the measured value may be used to determine whether the electrode is biased superior or inferior of one of the primum and secundum. Still further, the measured value is used to determine the orientation of the PFO tunnel relative to the catheter axis and/or the location, size, and orientation of one of the primum and the secundum. According to one aspect, selected ones of the plurality of electrodes are selectively activated such that only electrodes that address the PFO are activated.

A system for selectively delivering energy to tissue, including a multi-channel RF energy supply, wherein energy may be independently adjusted in at least two channels; a plurality of electrically independent electrodes, with at least one the electrode connected to each of the at least two channels such that energy applied to at least two electrodes may be independently controlled; a controller communicating with the multi-channel RF energy supply and controlling the delivery of energy to the electrodes, the controller measuring at least on of impedance and electrocardiac conductivity between a given pair of electrodes and adjusting the amount and manner in which energy is delivered in accordance with the measured value. The system may further include a plurality of thermocouples proximate the plurality of electrodes; wherein the controller receives a temperature signal from the thermocouples and terminates the delivery of energy to select ones of the plurality of electrodes in accordance with the measured temperature. According to one aspect, at least one of the plurality of electrodes includes a flange portion depending from a lower surface of the energy delivery device and configured to pierce or displace a surface of the tissue and the thermocouple is positioned to measure the temperature of the displaced or pierced tissue. According to another aspect, the flange defines a vent proximate to the thermocouple for venting steam from the tissue. Optionally, the controller measures at least one the impedance and electrocardiac conductivity between a plurality of different pairs of electrodes and independently adjusts the energy delivered to each of the plurality of different pairs of electrodes in accordance with the measured value.

A system for selectively delivering energy to tissue, including a multi-channel RF energy supply, wherein energy may be independently adjusted in at least two channels; a ground pad; a plurality of electrically independent electrodes, with at least one the electrode connected to each of the at least two channels such that energy applied to at least two electrodes may be independently controlled; a controller communicating with the multi-channel RF energy supply and controlling the delivery of energy to the electrically independent electrodes, the controller measuring at least one of impedance and electrocardiac conductivity between the ground pad and a selected one of the electrically independent electrodes and adjusting the amount and manner in which energy is delivered to the selected electrically independent electrode in accordance with the measured value.

A device for delivering energy to tissue, including an elongate flexible shaft having a proximal end and a distal end; at least one energy delivery device operably connected to the distal end of the shaft; a flange protruding from a tissue apposition surface of the energy delivery device; at least one thermocouple proximate the flange, whereby the flange is configured to pierce or displace tissue placed in abutment with the tissue apposition surface, and the thermocouple is configured to measure the temperature of the displaced or pierced tissue. According to one aspect, energy supplied to the at least one energy delivery device is terminated when the at least one thermocouple detects a threshold temperature. According to another aspect, the energy delivery device defines an aperture, the flange is formed proximate the aperture, and the thermocouple is mounted to the flange. The flange may include a sharpened portion configured to pierce tissue placed in abutment with the energy delivery device.

A method for sealing a patent foramen ovale, including providing a first electrode device on a first side of PFO tissues; providing a second electrode device on an opposing side of PFO tissues; exerting a force on the PFO tissues by bringing the first and second devices into abutment; and energizing at least one of the first and second electrode devices. The method may further including piercing the PFO tissue and threading one of the first and second electrode devices at least partially through the pierced PFO tissue. Moreover, one of the first and second electrode devices may include an expandable member threaded through the pierced PFO tissue. According to one aspect, the expandable member may include a balloon which is inflated after the expandable member is threaded through the pierced PFO tissue. Still further, one of the first and second electrode devices may serve as a return electrode and the other as an active electrode, the active electrode includes a plurality independent electrodes, wherein energy is individually supplied to the active electrodes, and the supply of energy to a given active electrode is terminated when one of an impedance and electrocardiac conductivity measured between the given electrode and the return electrode reaches a predefined threshold.

In the aforementioned method, the energy may be supplied for a predefined amount of time after the measured value reaches the predefined threshold, wherein the predefined threshold may be determined in relation to one of an initial impedance and initial electrocardiac conductivity for the given electrode. According to one aspect, the first and second electrodes are placed on opposing sides of the PFO tissue without piercing the PFO tissue. For example, the first and second electrode devices may be placed on opposing sides of the PFO tissue by threading one of the first and second electrode devices at least partially through the PFO tunnel. Still further, force is exerted on the PFO tissues by exerting a pulling force on one of the first and second electrode devices and a pushing force on the other of the first and second electrode devices. Alternatively, force may be exerted on the PFO tissues by exerting a pushing force on both the first and second electrode devices.

### BRIEF DESCRIPTION OF THE DRAWINGS

FIG. 1 is a diagram of the heart showing the foramen ovale;

FIGS. 2A and 2B are diagrams of a PFO-treatment apparatus according to the present invention;

FIGS. 3A-3F depict a first embodiment of an energy delivery device according to the present invention;

FIGS. 4A-4G are variations of the energy delivery device of FIGS. 3A-3F;

FIGS. 5A-5C are views of a second embodiment of an energy delivery device according to the present invention;

FIG. 6 is a third embodiment of an energy delivery device according to the present invention;

FIGS. 7A and 7B is a fourth embodiment of an energy delivery device according to the present invention;

FIGS. 8A-8K is a fifth embodiment of an energy delivery device according to the present invention;

FIGS. 9A-9D is a sixth embodiment of an energy delivery device according to the present invention;

FIGS. 10A and 10B is a seventh embodiment of an energy delivery device according to the present invention; and

FIGS. 11A-11C is an eighth embodiment of an energy delivery device according to the present invention.

### DETAILED DESCRIPTION OF THE INVENTION

The present invention relates to device used to coagulate, ablate tissue and/or weld tissue defects. Many of the methods and examples provided in this application relate to the treatment of cardiac defects such as patent foramen ovale (PFO); however, the utility of the device is not limited to the treatment of cardiac tissue.

The phrase “tissues adjacent a PFO,” or simply “PFO tissues,” for the purposes of this application, means tissues in, around or in the vicinity of a PFO which may be used or manipulated to help close the PFO. For example, tissues adjacent a PFO include septum primum tissue (“primum”), septum secundum tissue (“secundum”), atrial septal tissue inferior or superior to the septum primum or septum secundum, tissue within the tunnel of the PFO, tissue on the anterior atrial surface or the posterior atrial surface of the atrial septum and the like. The PFO tunnel refers to the opening or passageway between the right and left atrium resulting from non-union between the primum and secundum.

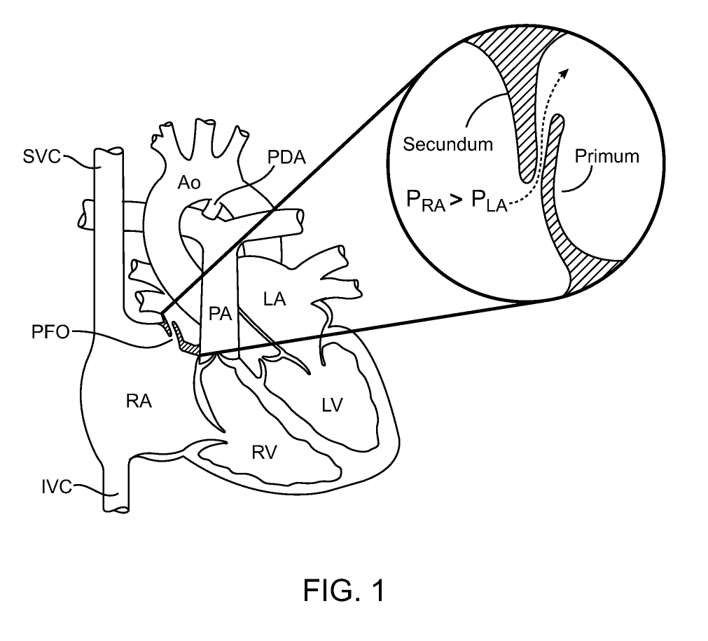

Devices of the invention generally include a catheter device having a proximal end and a distal end and at least one energy delivery device adjacent the distal end for applying energy to tissues adjacent the PFO. As mentioned above in the background section, FIG. 1 is a diagram of the heart showing the foramen ovale, with an arrow demonstrating that blood passes from the right atrium to the left atrium in the fetus. After birth, if the foramen ovale fails to close (thus becoming a PFO), blood may travel from the right atrium to the left atrium or vice versa, causing increased risk of stroke, migraine and possibly other adverse health conditions, as discussed above.

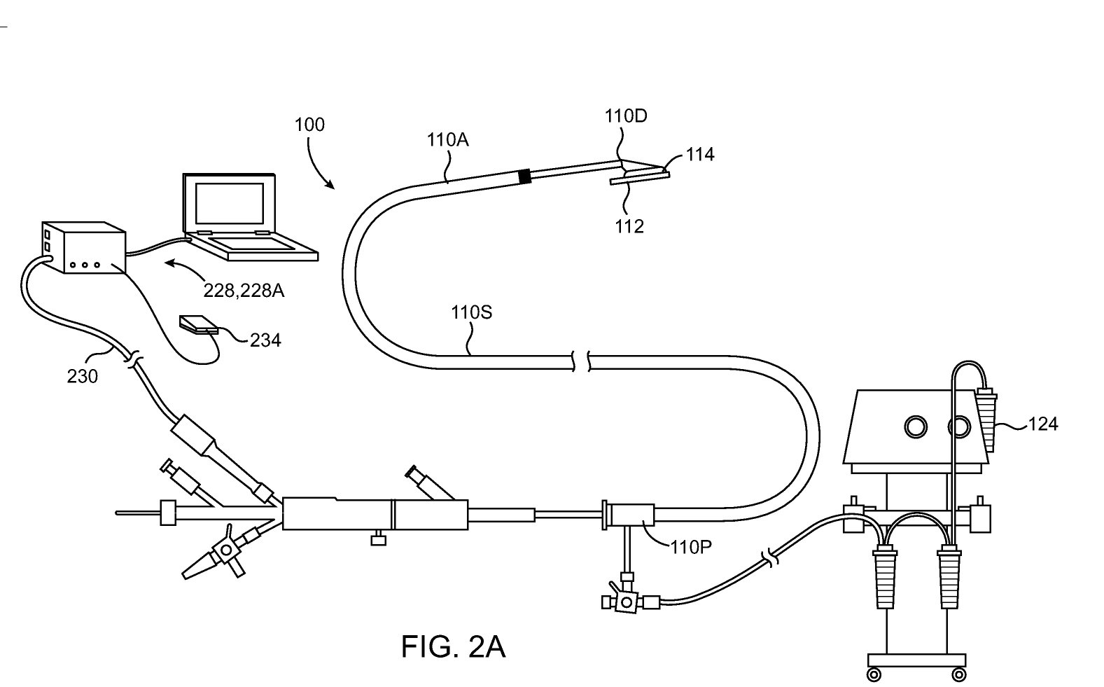

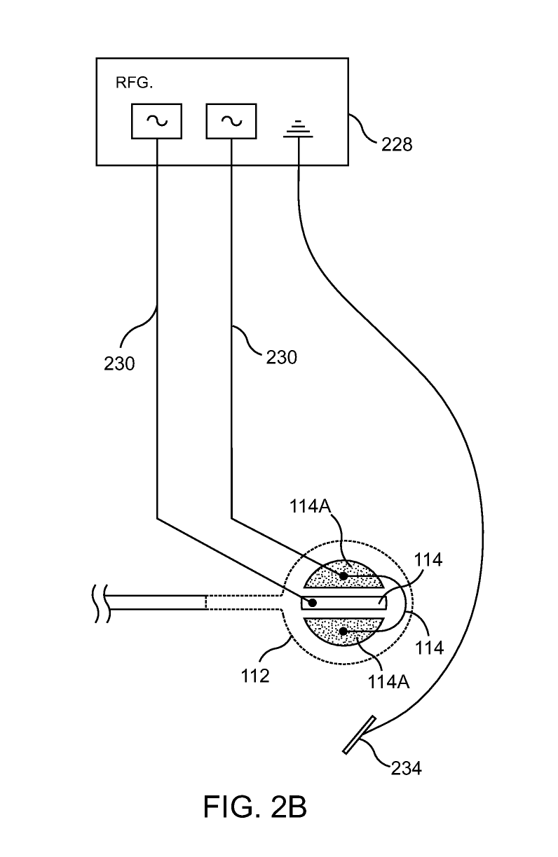

With reference to FIG. 2A, a PFO-treatment apparatus 100 of the present invention may be advanced through the vasculature of a patient to a position in the heart for treating a PFO. In this embodiment, apparatus 100 includes an elongate catheter device 110 which includes an elongate flexible shaft 110A having a proximal end 110P, a distal end 110D, and a sheath or sleeve 110S disposed over at least a portion of the flexible shaft. The depicted embodiment includes a distal housing 112 at or near distal end 110D. At least one energy transmission member(s) 114 may be positioned within or integrally formed with the distal housing 112, or may be positioned adjacent the housing 112. Still further, the energy transmission members 114 may be movable relative to the distal housing 112.

The distal housing 112 may be connected with a remote source of partial vacuum 124 via a vacuum lumen disposed within the catheter device 110 to bring the PFO tissues into apposition. In operation the distal housing 112 is placed in contact with the treatment area, a partial vacuum force (suction) is transmitted by the remote source of partial vacuum 124 via the vacuum lumen pulling the septum primum and septum secundum (PFO tissues) into apposition with each other as well as into apposition with the energy transmission member(s) 114.

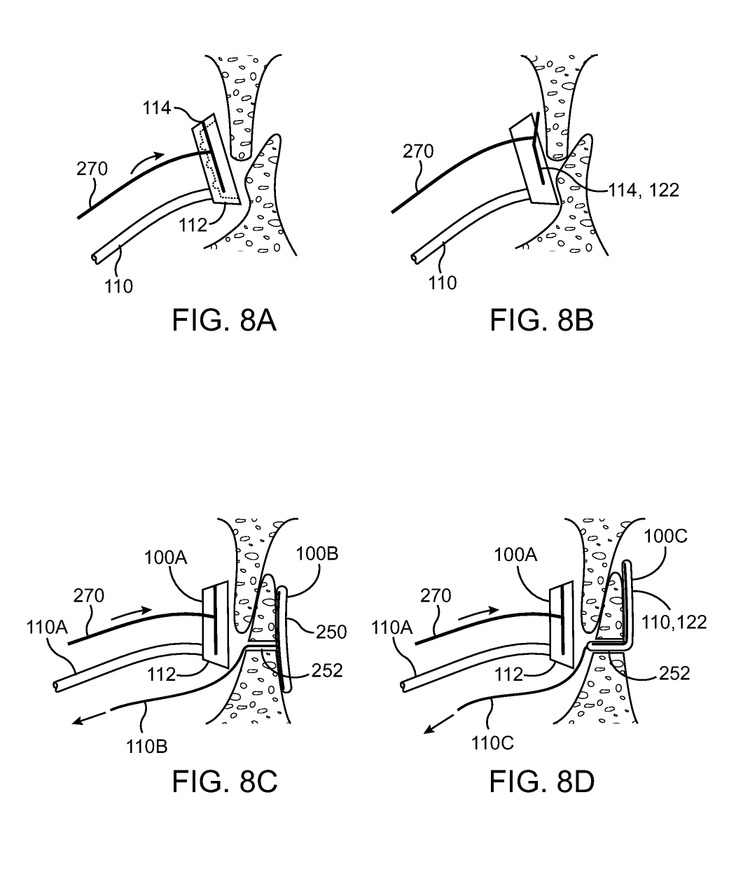

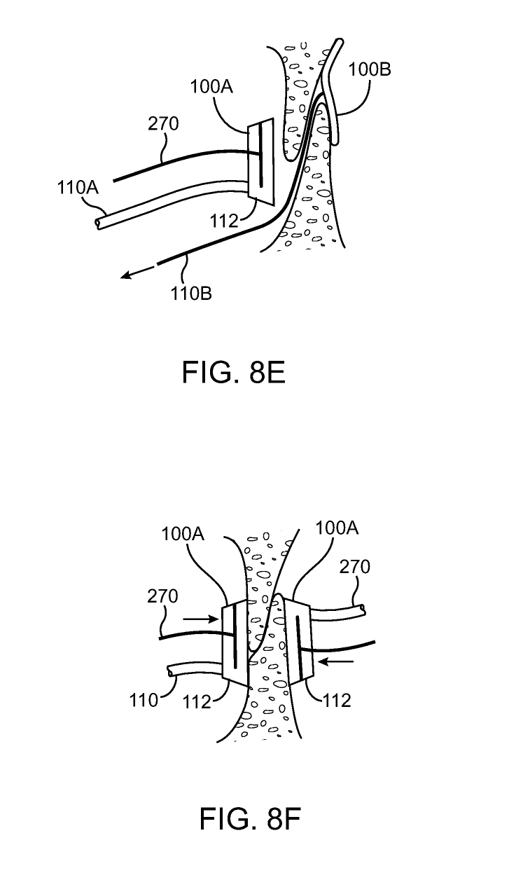

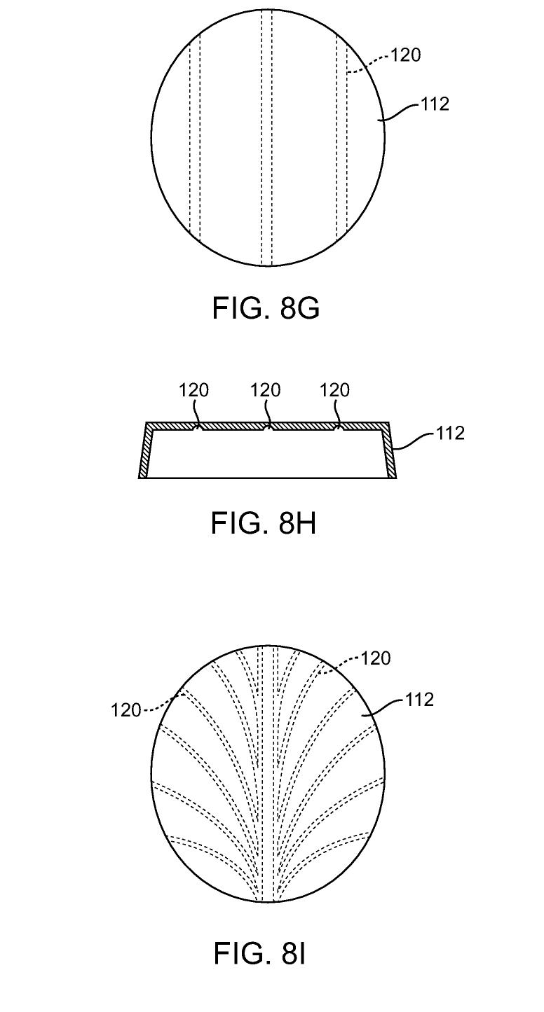

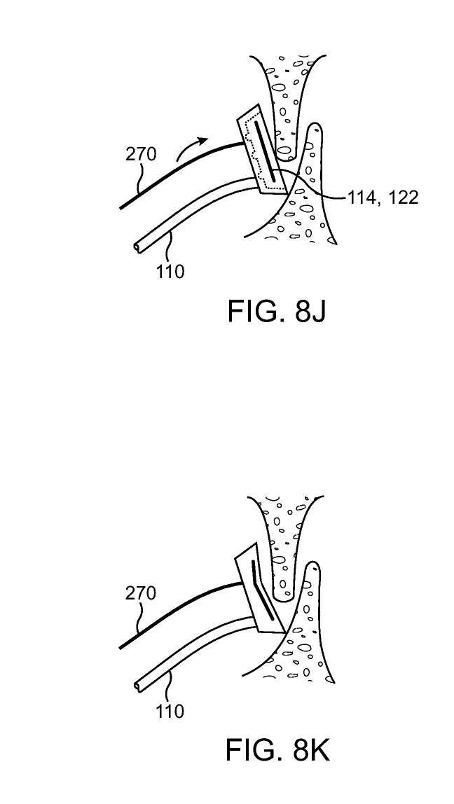

The distal housing 112 in all of the embodiments disclosed in this application may include one or more areas of reduced thickness 120 (FIG. 8xx) to promote the deformation of the distal housing 112 and/or assist collapsing the distal housing so that it may be inserted into the sheath or sleeve 110S.

Although the embodiment in FIG. 2A and many of the embodiments described herein below include one or more tissue apposition members such as the distal housing 112, devices of the present invention do not require such members. In some embodiments, the catheter device 110 may omit the distal housing 112 and/or other components designed for bringing the tissues together. Likewise, a device 100 according to the invention may employ a tissue apposition mechanism which does not rely on vacuum technology. Therefore, although much of the following discussion focuses on embodiments including tissue apposition members and the like, such members are not required and such limitations should not be read into the claims.

The energy transmission members 114 may be any means or mechanism for heating tissue such as but not limited to electrodes, RF electrodes, ultrasound transducer, microwave, patch antennas, dipole antennas, high or low current generators, or heating elements, i.e., resistive heating elements. While many of the illustrative examples disclosed herein refer to RF electrodes 114, the invention is not limited to RF electrodes.

As best seen in FIG. 2B the energy transmission members 114 are connected to a generator 228 via conductors 230. If the energy transmission members 114 are RF electrodes then the generator 228 is an RF generator. Correspondingly, if the energy transmission members 114 are resistive heating elements then the generator may be a current source. Reference to RF generator or generator 228 should be understood to include a current source suitable for use with electrodes, resistive heating elements or the like.

As will be explained below, the generator 228 may be provided with two or more independent channels and it may be desirable to connect transmission members 114 to one or the other of the separate channels to independently control the rate of the weld formation and/or control the location of the weld/lesion. Therefore, separate conductors 230 may be used to couple energy transmission members 114 with the discrete channels of the generator 228. “Channel” refers to independently adjustable power sources which enable the user to control the manner and amount of energy supplied. Connecting electrodes 114 to different channels of the generator 228 enables individual control of the power supplied to the electrodes 114.

The terms electrode and electrode segment (“segment”) as used throughout this application have different meanings. As used herein an electrode includes at least one segment but may include two or more electrically coupled segments. Since all segments of a given electrode are electrically coupled, energy applied to one segment flows to all of the coupled segments. In contrast, electrodes may be electrically independent of one another, or they may be electrically coupled. The electrodes may be coupled by a resistive voltage or current divider, capacitive coupler, inductive coupler, magnetic coupler or the like.

The energy transmission members 114 may be operated sequentially or in unison in a variety of different modes, as will be explained below in further detail. An optional ground pad (dedicated return electrode) 234 (FIGS. 2A and 2B) connected to the ground of the generator 228 may be electrically coupled to the patient, e.g., using a conductive adhesive as known in the art. The ground pad 234 may be placed in contact with the patient's skin at a location generally remote from the energy transmission members 114 or at any convenient location on or in the patient. In some embodiments one of the electrodes 114 may serve as a return electrode.

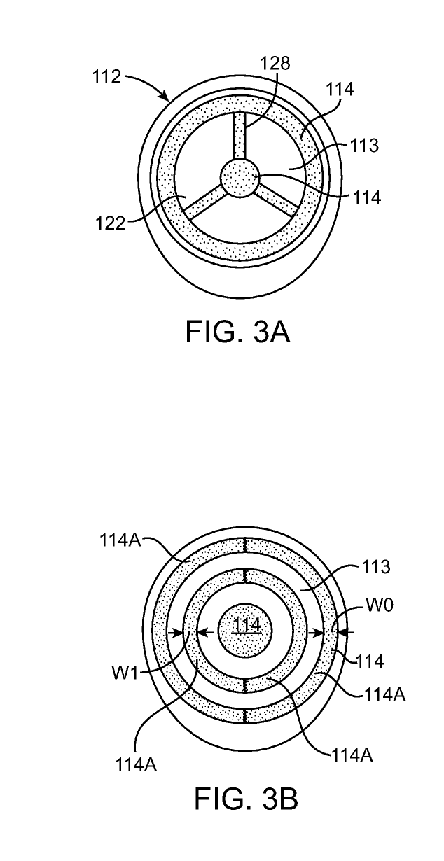

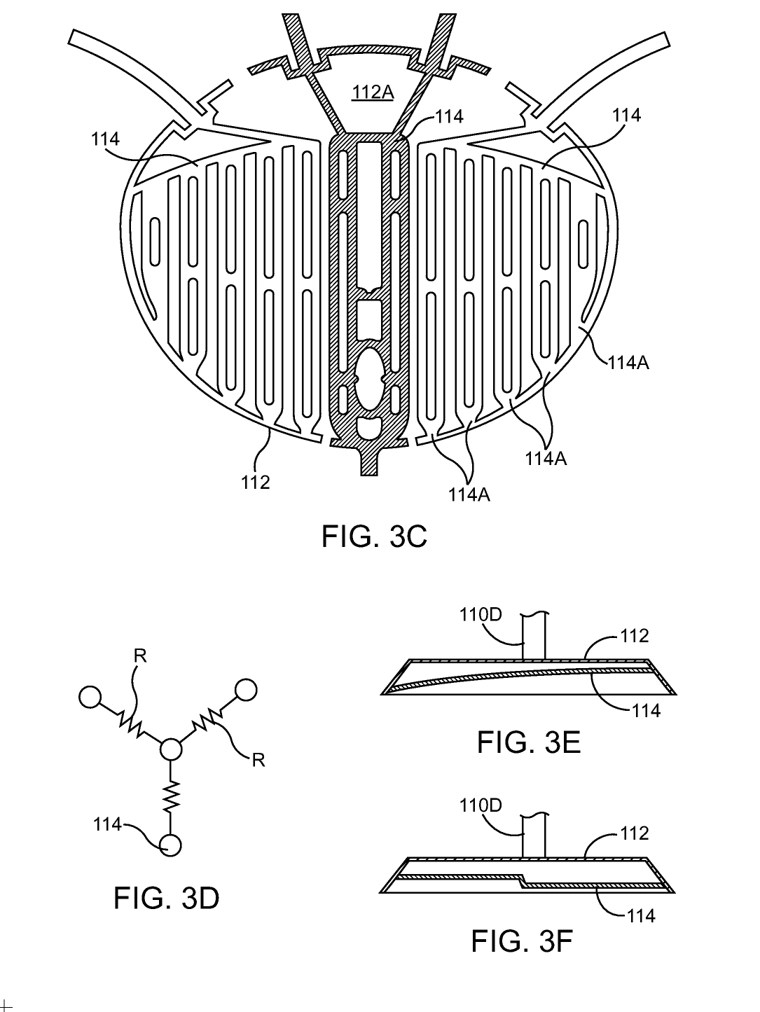

FIG. 3A is an enlarged bottom view of the distal end 110D of the PFO apparatus 100 illustrating a first embodiment of the energy transmission members 114 of the present invention. The energy transmission member(s) 114 may be mounted on an inner surface 112A of the distal housing 112, may be integrally formed with, e.g., molded into, the distal housing 112, or they may be mounted on a substrate 122, or they may simply be free movably independent structures. The electrodes 114 may be integrally formed with the substrate 122, and the substrate 122 may be affixed or mounted within the housing 112. In any event the electrodes 114 are attached to a distal end 110D of the flexible shaft 110A.

The energy transmission members 114 have a tissue apposition surface adapted to contact the tissue to be treated. The tissue apposition surface of the energy transmission members 114 may be generally planar, but the energy transmission members 114 may have a non-coplanar tissue apposition surface configured to match or fit the tissue anatomy. For example, the PFO tissue frequently includes a step or lip formed by a relatively thick secundum and a relatively thin primum. FIGS. 3E and 3F depict a side view of a non-coplanar energy deliver device 114. More particularly, FIG. 3E depicts an energy deliver device 114 having a stepped profile whereas FIG. 3F depicts energy deliver device 114 having a curved profile.

As will be described in detail below, structural members such as struts 128 may be used to support the energy transmission members 114 such that they generally maintain a fixed relationship relative to one another while still allowing the individual energy transmission members 114 to conform to the tissue anatomy. The energy transmission members 114 and the distal housing 112 cooperatively define gaps or passages 113 in communication with the vacuum lumen (not illustrated) to facilitate the transmission of suction from the source of partial vacuum 124 to the tissue.

The struts 128 are preferably formed from a non-conductive or poorly conductive material so as to maintain the electrical isolation among the energy transmission members 114. FIG. 3D is a functional drawing of the energy delivery device 114 including poorly conductive struts 128 which are depicted as resistors R.

Depending on the resistive value, the struts 128 resistor(s) may serve as a structural member, or both as a structural member and as a conductive pathway. Notably, at the power levels typically supplied by RF generator 228 (e.g. 100 W), a 1 mega ohm resistor R will not allow an appreciable amount of current to flow and the resistor R will primarily serve as a structural member. In contrast, a 5 ohm resistor R will allow current to flow between the electrodes 114 and will also serve as a structural member to maintain the spacing between two interconnected electrodes 114.

The distal housing 112 and the substrate 122 (if used) are preferably formed of a flexible (resilient), nonconductive or poorly conductive material. For example, the distal housing 112 may be formed of plastic or silicon and the substrate may be formed of plastic, silicon or metal, e.g., a nickel titanium alloy such as Nitinol®. If the substrate is formed of metal it may include an electrically insulating coating to preserve electrical isolation of the energy transmission members 114.

FIG. 3A illustrates an embodiment including two electrically independent concentric electrodes 114. According to one embodiment, the second electrode 114 is electrically independent the first electrode 114. However, if desired, both of the electrodes 114 may be electrically connected to act as a single electrode (having two segments). In the depicted embodiment the second electrode 114 at least partially surrounds and is spaced apart from the first electrode 114.

The first electrode 114 may be circular. The second electrode 114 may be elongated, and may form a ring concentric with the first electrode 114.

According to one embodiment both electrodes 114 are connected to the same channel of the generator 228. According to another embodiment each electrode 114 is connected to a different channel of the generator 228 such that the application of energy may be independently controlled for each electrode 114.

FIG. 3B illustrates a version of the distal housing 112 which includes three concentric energy transmission members 114. Preferably each electrode 114 is connected to a separate channel of the generator 228. However, if desired, two or more electrodes 114 may be connected to the same channel of the generator 228. For example, the innermost and outermost electrodes 114 may be connected to the same channel. Moreover, two or more of the electrodes 114 may be electrically shorted proximate the distal end 110D of the elongate flexible shaft 110A thereby eliminating the need for one or more conductors 230. For example, the innermost and outermost electrodes 114 may be shorted. Shorting two electrodes 114 results in the electrodes acting as electrically coupled segments of a single electrode 114.

The width and/or surface area of each electrode 114 may differ. Notably, the relative size/shape of the electrodes may be selected to control the density of energy delivered to the tissue. Empirical evidence indicates that it is difficult to obtain uniform heating with a single large electrode 114, and that it is therefore preferable to use several smaller electrodes. In the depicted embodiment, the width W0 of the outermost electrode is smaller the width W1 of the intermediate electrode 114. The primary consideration in selecting the size and geometry of the electrode is to deliver an appropriate energy density in order to achieve the desired tissue effect (tissue welding, tissue tightening) without causing deleterious effects to the tissue.

The energy transmission members 114 may be operated in a unipolar (monopolar) mode by applying a voltage source from the generator 228 to the treatment site through the energy transmission member 114, causing an electrical current to flow through the tissue to the ground pad 234 and then back to the generator 228.

A controller 228A (FIG. 2A) within the generator 228 enables the operator to apply electrical current in various combinations to the transmission members 114. For example, current may be applied simultaneously to each of the transmission members 114, sequentially to one transmission member 114 at a time, or in a step-wise fashion with current applied to one transmission member 114 for a first period and then to two transmission members 114 for a second period, and then to three transmission members 114 for a third time period. Likewise, one or more transmission members 114 may be operated in a monopolar mode for a first time interval and then the same or other transmission members 114 may be operated in a bipolar mode or a multipolar mode as described below, or the bipolar mode could precede the monopolar mode.

Each energy transmission member 114 may be divided into two or more electrically coupled segments 114A (FIGS. 2B and 3B). The segments 114A of the electrode 114 may be independently movable or independently conformable to facilitate conformance of the electrode 114 with the tissue anatomy. Splitting energy transmission member 114 into multiple segments 114A may make it easier to collapse the energy transmission member 114 into the catheter 110. FIG. 3B illustrates a version in which the middle and second energy transmission members 114 are divided each divided into segments 114A. It should be appreciated that given transmission members 114 segment may be divided into as many segments 114A as desired. The use of multiple segments 114A has minimal if any impact on energy delivery. In contrast, the use of multiple relatively small electrodes 114 rather than a single large electrode has a significant impact on energy delivery because the smaller electrodes have a greater energy density and are able to deliver energy more uniformly than a large electrode.

In the bipolar mode, the polarity of the electrodes 114 alternates, with one of the electrodes 114 serving as the return electrode.

According to one embodiment the controller 228A controls which electrode 114 is the return electrode. Thus, the controller 228A may “steer” the lesion/weld formation by changing which electrode(s) 114 are active and which electrode serves as the return electrode 114, in monopolar mode or other 114, 114A in bipolar mode.

The apparatus 100 may further be operated in a “multipolar” mode which is a hybrid between the monopolar and bipolar modes of operation. In the multipolar mode of operation, differing voltage levels are supplied to two or more electrodes. The multipolar mode of operation will now be explained with reference to FIG. 3A. Let us assume that the voltage supplied to the first electrode 114 is greater than the voltage supplied to the second electrode 114. As in a conventional monopolar mode, current flows from the first and second electrodes 114, 114 through the tissue to the ground pad 234. However, because the first electrode 114 is at a greater potential than the second electrode 114, a portion of the current from the first electrode 114 will flow through the tissue to the second electrode 114 and then through the tissue to the ground pad 234. No changes in wiring are required to change the mode of operation; the same device 100 can function in different modes of operation as determined by the controller 228A.

In FIG. 2B, the distal housing 112 which includes two electrically independent electrodes 114. In the illustrated embodiment, the central electrode 114 is sandwiched or interleaved between two electrically coupled segments 114A of the second electrode 114. This concept may be expanded to include any number of interleaved segments 114A of any number of electrodes 114.

FIG. 3C is a slight variation on the distal housing 112 of FIG. 3B. The distal housing 112 in FIG. 3C includes three electrically independent electrodes 114. The illustrated electrodes 114 are generally rectangular in shape; however, the shape of the electrodes is not critical. In the illustrated embodiment, the central electrode 114 includes two electrically coupled segments 114A whereas the electrodes 114 on either side of the central electrode include five electrically coupled segments 114A; however, each of the electrodes may include any number of electrically coupled segments 114A. In the illustrated embodiment each of the segments 114A are generally the same size and shape, however, the invention is not limited to the illustrated embodiment. It should however be noted that the surface area of the central electrode is different from the electrodes on either side, yielding a different energy density in the central electrode. Thus a device having multiple electrodes 114 each having a different surface area would result in a different energy density for each electrode 114. The relationship between the size of the electrode and the energy density may be utilized to provide the appropriate energy density for each region of the treatment zone.

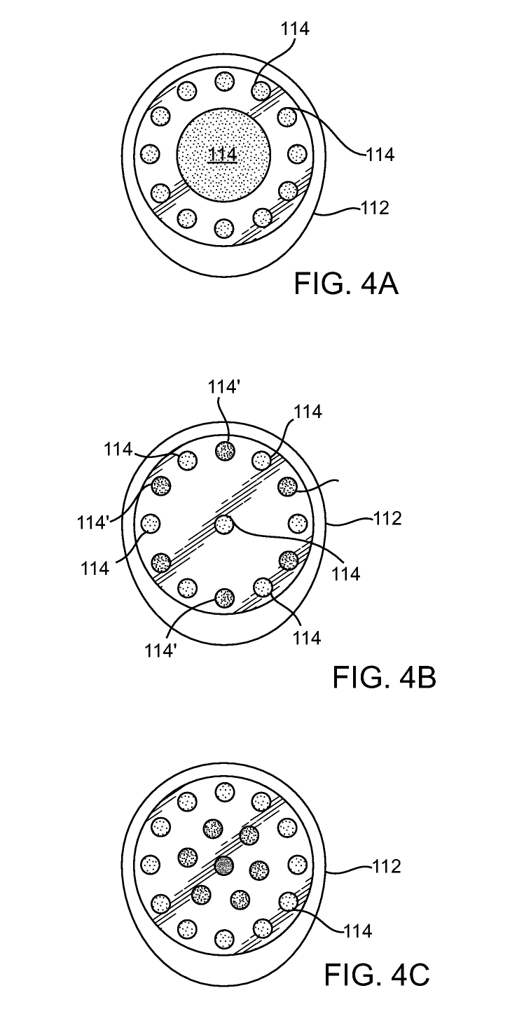

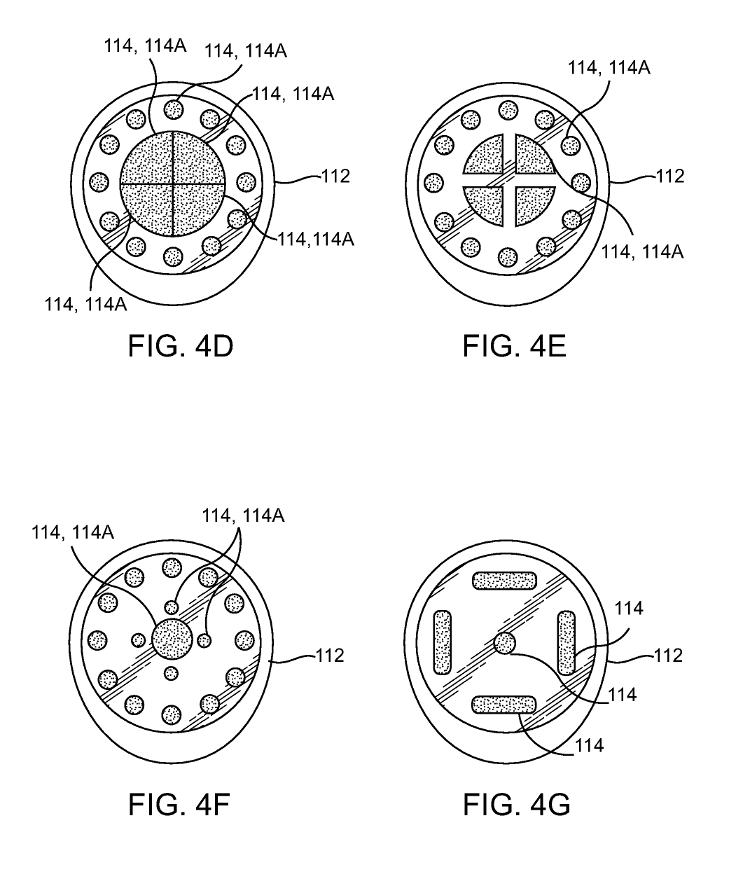

FIGS. 4A and 4B illustrates variations of the energy transmission member 114 including a central electrode 114 and a plurality of satellite electrodes 114. FIG. 4A depicts a distal housing 112 with a central electrode 114 having a larger surface area than the satellite electrodes, and FIG. 4B depicts a distal housing 112 with a central electrode 114 which is generally the same size as the satellite electrodes 112.

The satellite electrodes 114 may be spaced a uniform distance from one another. The satellite electrodes 114 may be formed along one or more radial distances from the central electrode. The central electrode 114 may have a greater surface area than the satellite electrodes. The central electrode 114 may be connected to a different channel of the generator 228 than the satellite electrodes 114.

The satellite electrodes 114 may be divided into two or more groups, with each group connected to a different channel of the generator 228. By manner of illustration, the electrodes 114 and 114′ in FIG. 4B are connected to a different channels of the generator 228.

Alternatively, all of the satellite electrodes 114 may be electrically coupled to form a single electrode 114. For example, electrodes 114 along a first radial distance from the central electrode may be connected to a first channel of the generator 228, and electrodes 114 along a second radial distance from the central electrode may be connected to a second channel of the generator 228 (FIG. 4C).

Alternatively, electrodes 114 along a given radial distance from the central electrode 114 may be divided into groups such that some are connected to a first channel of the generator 228 and others to a second channel of the generator 228 (FIG. 4B).

It should be noted that the invention does not require a central electrode 114. It should further be understood that electrodes 114 may be disposed at any number of radial distances, and that the electrodes 114 may be distributed non-uniformly with a dense concentration of electrodes in one area of the treatment zone and a sparse concentration of electrodes in another area. The electrodes 114 may be of different sizes. For example, it may be desirable to have a number of small electrodes which are closely spaced together in one area of the treatment zone (to provide a higher energy density) and a number of larger electrodes in another area. In FIG. 4C electrodes 114 are positioned along two radial distances from the central electrode 114.

As illustrated in FIG. 4D, the central electrode 114 may be replaced by two or more electrically independent electrodes 114 or electrically coupled segments 114A to facilitate the deployment of the device from the sheath 110S. In the illustrated embodiment, four electrodes 114 or segments 114A are provided. However, the invention is not limited to the illustrated embodiments.

The configuration of the energy deliver devices 114 in FIG. 4E is essentially identical to that shown in FIG. 4D, except that the central electrode(s) 114 or electrically coupled segments 114A are spaced slightly from one another. Preferably, each of the electrodes 114 or segments 114A possesses some degree movement relative to the other electrodes or segments to facilitate conformance of the electrodes to tissue anatomy. In the illustrated embodiment four wedge-shaped electrodes 114 or segments 114A are provided; however, the invention is not limited to any specific shape or number of electrodes 114 or segments 114A.

FIG. 4F illustrates another variation in which the central electrode 114 is divided into five electrodes 114 or electrically coupled segments 114A including a central segment (or electrode) and four satellite segments (or electrodes) formed a uniform radial distance from the central segment. The invention is not limited to the illustrated embodiments, and it is contemplated that the central electrode 114 may be divided into any number of segments (or electrodes).

FIG. 4G illustrates another variation including a central electrode 114 at least partially surrounded by plurality of shaped electrodes 114. In the illustrated embodiment the shaped electrodes 114 are elongate and generally straight; however, the shaped electrodes may assume any shape and may for example be curved or arcuate.

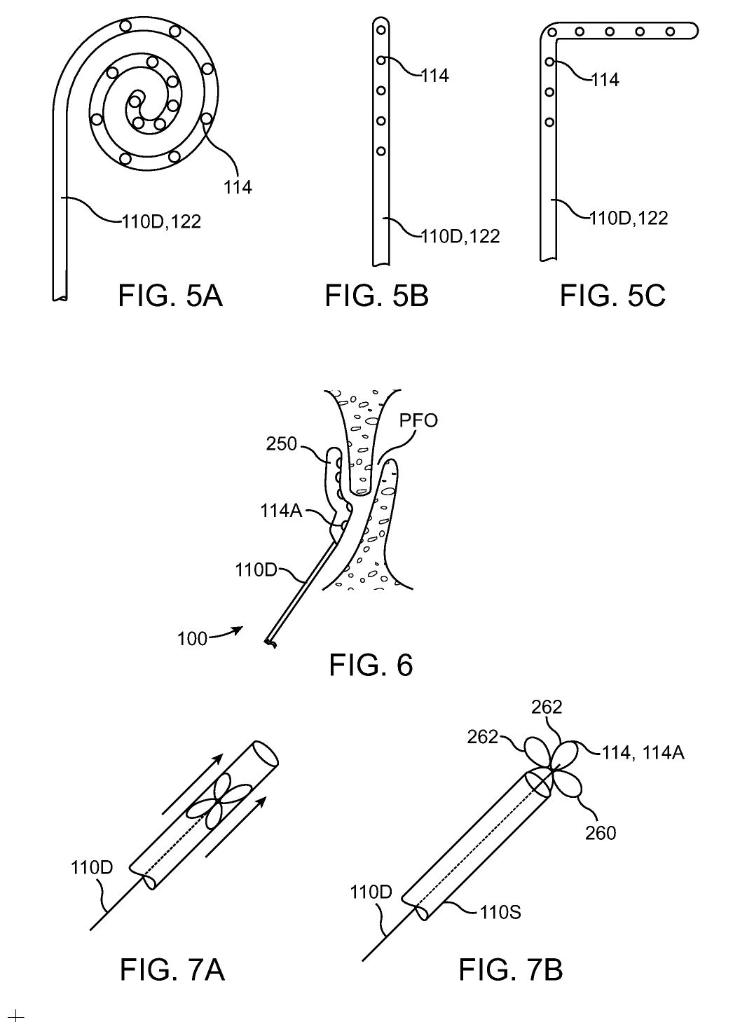

FIGS. 5A-5C depicts an alternate embodiment including a plurality of energy transmission members 114 formed on the distal end of the flexible shaft 110D or on substrate 122 attached to the shaft 110. The substrate 122 or distal end of shaft 110D may be elastically deformed from its native shape shown in FIG. 5A and FIG. 5C to a shape amenable for catheter-based delivery shown in FIG. 5B. The substrate 122 (or distal end of shaft 110D) resumes its native shape once it is no longer restrained, i.e., after the substrate 122 is deployed from the catheter 110. The substrate 122 may include a shape memory alloy such as NiTi (Nitinol®).

It should be noted that the embodiment depicted in FIGS. 5A-5C does not include distal housing 112; however, an appropriate distal housing 112 could be provided if desired. As shown in FIG. 5A the native state of the substrate 122 or distal end 110D is generally a helix, i.e., spiral-shaped; however, other shapes are contemplated. For example the substrate or distal end 110D could form an L-shape FIG. 5C, a square, or a series of interlocking squares or any other shape. The primary consideration in selecting the shape of the substrate 122 is the ease of collapsibility and deployment to/from the sheath 110S. However, additional considerations include the size and shape of the treatment area and the tissue anatomy e.g. whether the tissue is planar.

The energy transmission members 114 may be any of the embodiments disclosed herein. Moreover, the energy transmission members 114 may comprise circumferential bands disposed around the distal end of the flexible shaft 110D or on substrate 122 attached to the shaft 110.

As with the previously described embodiments, one or more of the transmission members 114 may be electrically independent. Likewise, the transmission members 114 may be operated in a variety of modalities (monopolar, bipolar, multipolar), and power may be applied simultaneously to all of the electrodes or in a step-wise or incremental manner. For example, power may first be applied to the centrally located transmission members 114 and power may subsequently be applied to the peripheral transmission members 114.

FIG. 6 illustrates a device 100 including one or more electrodes 114 formed on a conformal balloon 250. Like the previous embodiments, device 100 is preferably deployed to the treatment site using catheter 110. The balloon 250 is preferably deployed to the treatment site in a deflated or partially deflated state. Upon inflation the balloon 250 assumes its predefined conformal shape. While tissue anatomy varies, the secundum is generally thicker than the primum. The difference in tissue thickness sometime presents a distinct lip or step. The balloon 250 is configured to assume a shape which includes a complimentary step such that the electrode(s) 114 formed on the surface of the balloon 250 is/are placed in abutment with both the primum and secundum. The balloon 250 may include a single electrode 114 comprising multiple electrically coupled segments 114A, or may include two or more electrodes 114 each of which may include any number of electrically coupled segments 114A.

FIGS. 7A and 7B depict a device 100 in a collapsed and a deployed state. The device 100 includes a frame 260 formed of an elastically deformable material such as Nitinol® which resumes its native shape (FIG. 7B) once fully deployed from the catheter 110. In addition to serving as a structural member, the frame 260 may serve a dual purpose as an electrode 114. Alternatively, one or more electrodes 114 may be formed on the frame 260. Again, each electrode 114 may include any number of electrically coupled segments 114A. In the embodiment depicted in FIG. 7B, the frame 260 includes a plurality of flower-like portions 262. Preferably, each portion 262 is highly flexible such that each portion 262 may independently conform to the tissue anatomy. Each portion 262 may constitute a separate electrode 114. Alternatively, two or more portions 262 may cooperatively form a single electrode 114.

FIGS. 8A and 8B depict a device 100 which includes a distal housing 112, a deformable electrode 114 and a pusher 270. The pusher 270 is an elongate member such as a guidewire or the like capable of transmitting force. A distal end of the pusher 270 is operably connected to the electrode(s) 114 or to substrate 122 on which the electrode(s) 114 is/are attached and a proximal end of the pusher 270 is manipulated (pulled/pushed) by the user to deflect the electrode 114. The electrode 114 may be any of the embodiments described herein, and may include a single electrode 114 (which may include multiple segments 114A) or multiple electrically isolated electrodes 114. The electrode 114 may comprise two electrically coupled segments 114A with the pusher 270 operably connected to one segment 114A such the user can move the one segment relative to the other. The two segments 114A may be connected by a living hinge, e.g. a thinned or scored portion of the electrode 114. Alternatively, the electrode 114 may include two electrically isolated electrodes 114 with the pusher 270 operably connected to one electrode 114 such the user can move the one electrode 114 relative to the other.

The electrode 114 may be deformable. The pusher 270 is connected to a proximal side of the electrode 114 such that the user elastically deforms the electrode 114 into conformance with the tissue anatomy by manipulating the pusher 270. The electrode 114 and/or the distal housing 112 may include one or more areas of reduced thickness 120 to promote the deformation of the electrode 114.

In operation, the electrode 114 is operably attached to the distal end of the catheter 110D and is deployed to the treatment site through sleeve 110S. In some embodiments the electrode 114 is connected or integrally formed with the distal housing 112 which is attached to the distal end of the catheter 110D. The pusher 270 is operably connected to the electrode 114 or the substrate 122 on which the electrode 114 is mounted.

The device 100 of FIG. 8A may be used in conjunction with another the device to squeeze the PFO tissue flaps into apposition. FIG. 8C illustrates how the device 100 of FIG. 8A may be used in combination with the device 100 of FIG. 6, and FIG. 8D illustrates how the device 100 of FIG. 8A may be used in combination with the device 100 of FIG. 5C.

FIG. 8C illustrates an approach in which device 100A is used to push from one side of the heart, and a device 100B threaded through a puncture 252 made in the PFO tissue is used to pull the PFO tissue into abutment with device 100A. The puncture 252 may be made in either/both the primum and/or the secundum; however, the FIG. 8C illustrates a puncture made in the primum. The device 100B includes an expandable member 250 which may be a balloon or the like. The member 250 is preferably transported through the puncture 252 in its deflated state and then inflated.

The device 100A may be positioned on either the right or left atria with the device 100B on the opposing atrium. Still further the PFO may be approached from either the left or the right atria; however, the preferred approach is from the right atrium.

FIG. 8D illustrates an approach in which device 100A is used to push from one side of the heart, and a device 100C threaded through a puncture 252 made in the PFO tissue is used to pull the PFO tissue into abutment with device 100A. Again, the puncture 252 may be made in either or both of the primum and/or the secundum; however, the FIG. 8D illustrates a puncture made in the primum. The device 100C includes one or more transmission members 114 formed on the distal end of the flexible shaft 110D or on substrate 122 attached to the shaft 110. The substrate 122 or distal end of shaft 110D may be elastically deformed from its native shape shown in FIG. 5A and FIG. 5C to a shape amenable for catheter-based delivery shown in FIG. 5B. The substrate 122 (or distal end of shaft 110D) is passed through the puncture 252 whereupon it resumes its native shape.

Again, the device 100A may be positioned on either the right or left atria with the device 100C on the opposing atrium. Still further the PFO may be approached from either the left or the right atria; however, the preferred approach is from the right atrium. However, the presently preferred approach is to approach from the right atrium, and position the device 100C from the right atrium into the left atrium.

FIG. 8E illustrates an approach in which device 100A is used to push from one side of the heart, and a device 100B or a device 100C is threaded through the PFO tunnel, i.e. the tunnel between the left and right atria formed by the non-union of the PFO tissue. The user pulls the PFO tissues into apposition by pushing on the device 100A and pulling on the device 100B or 100C.

FIG. 8F illustrates an approach in which device 100A is used to push from one side of the septum and another device 100A is issued to push from the opposing side of the septum. More particularly, one device 100A is threaded into the left atrium and another device 110A is threaded into the right atrium without piercing the septum. The surgeon brings the PFO tissue into apposition by pushing the two devices 100A into apposition.

Each of the devices of the present invention may be operated in any of a number of different modes, e.g., monopolar, bipolar, or multipolar. With respect to the embodiments depicted in FIGS. 8C-8F, one device 100A, 100B, 100C may serve as the active electrode and the other device 100A, 100B, 100C may serve as the return electrode. For example in FIG. 8C device 100A may include one or more active electrodes 114 and device 100B may include one or more return electrodes, or vice versa.

FIG. 8G is a top view of a distal housing 112 including one or more scores or areas of diminished thickness 120 which facilitate deformation of the housing 112 and/or collapsing/deployment of the housing 112 to/from the sleeve 110S. FIG. 8H is a side view of 8G. FIG. 8I shows a slight modification of FIG. 8G which is provided to illustrate that the score marks or areas of diminished thickness 120 to be provided in any number of different orientations. The areas of diminished thickness 120 depicted in FIGS. 8G-8I and variations thereof may be incorporated into the distal housing 112 of any of the embodiments contained in this disclosure.

FIGS. 8J and 8K depict a device 100 which, except for the location of the distal end of the pusher 270, is identical to device 100 of FIGS. 8A and 8B. This same modification may be incorporated into the devices depicted in FIGS. 8C-8F but such drawings have been omitted for the sake of brevity. In device 100 according to FIG. 8J the distal end of the pusher 270 is operably connected to the distal housing 112. The user manipulates the proximal end of the pusher 270 in order to deflect the housing 112 and indirectly deforms the electrode 114. The electrode 114 and/or the distal housing 112 may each include one or more areas of reduced thickness 120 (FIGS. 8G-8I) or a score e.g., a living hinge, to promote the deformation of the distal housing 112 and/or the electrode 114.

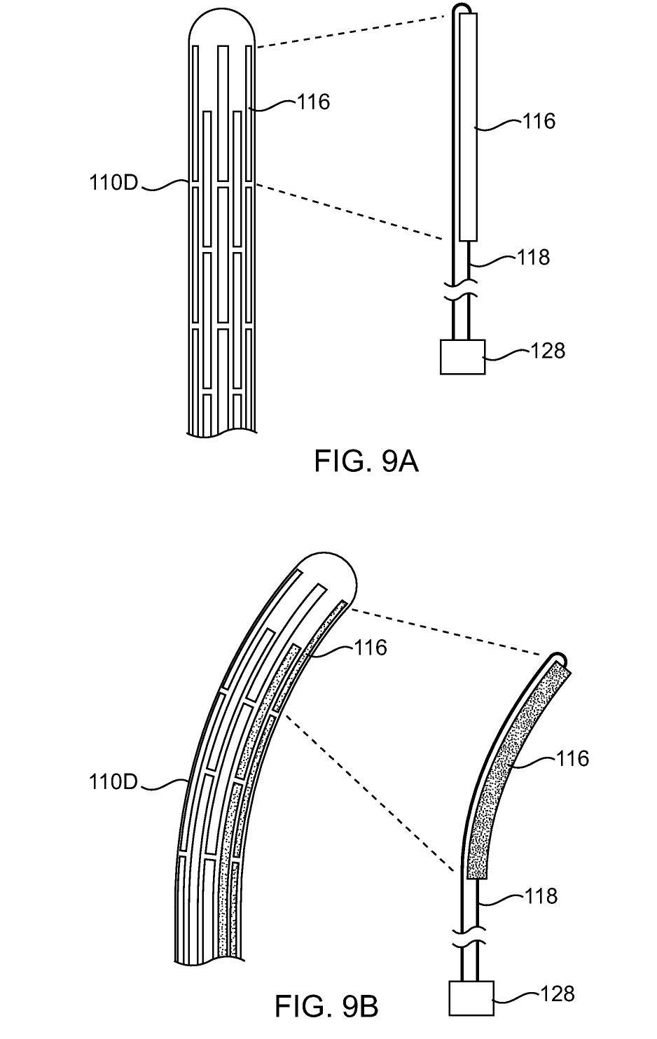

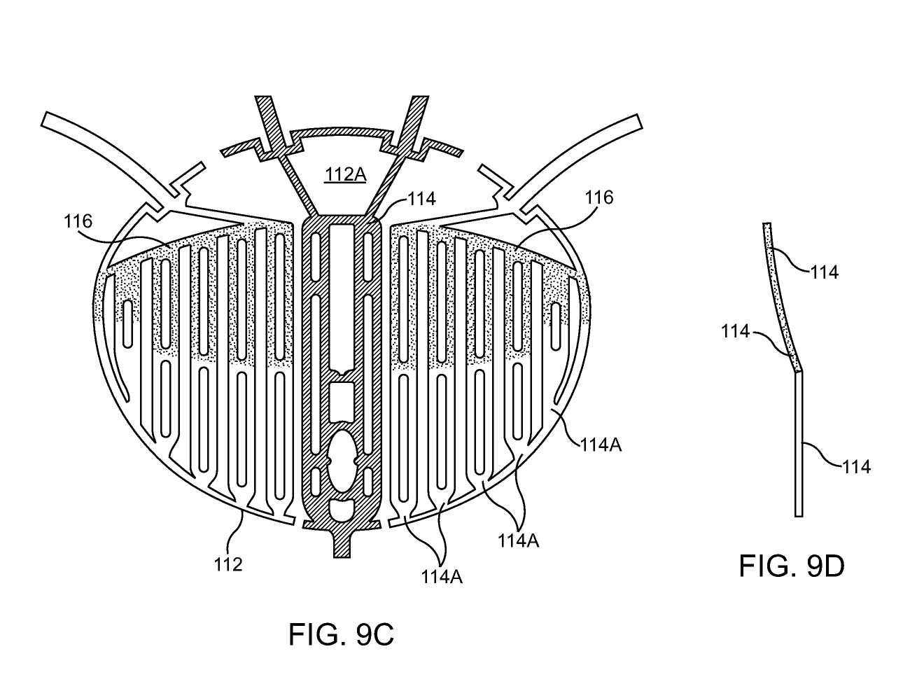

FIGS. 9A-9E depict a device 100 which is deployed to the treatment site using catheter 110 like the previously described embodiments, and includes at least one RF electrode 114. The electrode 114, substrate 122, and/or the distal end 110D of the catheter may be configured to deform (bend) when heated past a transition temperature. The angular orientation of the distal end 110D and/or the electrode 114 may be modified in situ by providing one or more discrete selective adjustment zones 116 which have an initial shape when deployed to the treatment area but which resume a native shape or orientation when heated past a transition temperature. By employing multiple independently adjustment zones 116 the electrode may be customized in situ to assume any number of complex shapes. Heating of the adjustment zone 116 may be accomplished in situ, for example, by resistive heating action as current is supplied to the distal end 110D and/or electrode 114. The electrodes 114 may be any of the electrodes described in this disclosure.

The adjustment zone 116 may be made of a nickel titanium alloy and configured to contract like muscles when electrically driven. This ability to flex or shorten is a characteristic of certain alloys, which dynamically change their internal structure at certain temperatures. Nickel titanium alloys contract by several percent of their length when heated and can then be easily stretched out again as they cool back down to room temperature. Like a light bulb, both heating and cooling can occur quite quickly. The contraction of Nickel Titanium (Nitinol® or Flexinol®) wires when heated is opposite to ordinary thermal expansion, and may exert a relatively large force for its small size. Movement occurs through an internal “solid state” restructuring in the material.

The substrate 122, distal end 110D and/or electrode 114 may include one or more adjustment zones 116 which enable the user to selectively adjust the orientation and/or geometry of the distal end 110D and/or electrode 114 by heating the appropriate adjustment zone 116. In this manner the user can steer the electrode 114 and/or adjust the electrode 114 to match the tissue anatomy.

FIGS. 9A and 9B show the distal end 110D before and after the adjustment zone has been heated past the transition temperature. A heating device 118 such as a resistive element or the like may be provided proximate the adjustment zones 116 to heat the adjustment zones 116 above the transition temperature. The heating device 118 depicted in FIGS. 9A and 9B is an insulated wire through which high frequency alternating current or direct current is sent to heat the adjustment zones 116 above the transition temperature for flexing.

FIGS. 9C-9E depict a distal housing 112 in which the electrode 114 is also the adjustment zone 116 and/or the heating device 118, or a discrete adjustment zone 116 and/or discrete heating device 118 are mounted/bonded to the electrode 114. The electrode 114 may also serve as the heating device 118 which is mounted to a discrete adjustment zone 116 (which may be the substrate 122).

The electrode 114 may serve as both the adjustment zone 116 and the heating device 118. In such case it may be desirable that the electrode 114 stay below the transition temperature in normal operation. If the user elects to actuate the adjustment zone 116 he/she merely increases the current supplied to electrode 114.

The electrode 114 may also serve as the adjustment zone 116 which is mounted to a discrete heating device 118 (which may be the wires depicted in FIGS. 9A and 9B).

FIG. 9C is a top view of the distal housing 112 which may include (but is not limited to) any of the embodiments disclosed herein, before the adjustment zone 116 is actuated. FIG. 9D shows a side view of the distal housing 112 before the adjustment zone 116 is actuated. FIG. 9E shows a side view of the distal housing 112 after the adjustment zone 116 is actuated.

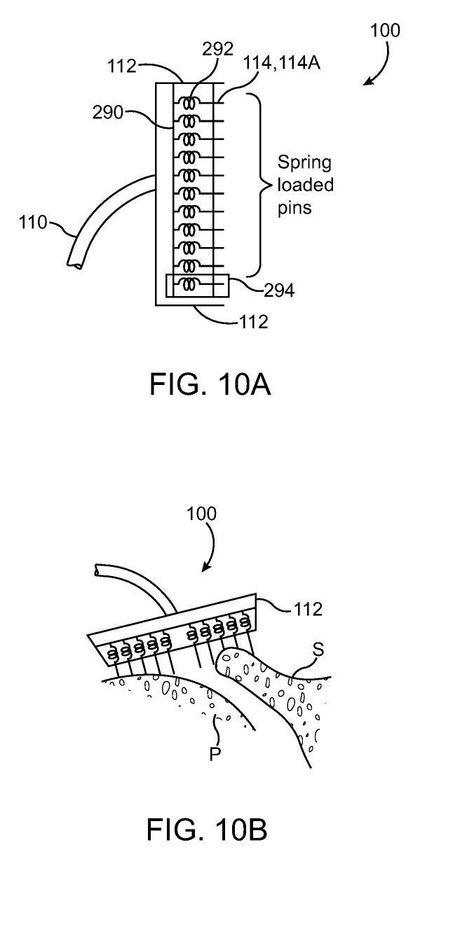

FIGS. 10A and 10B depict a device 100 which, like the above-described embodiments, may be deployed to the treatment site using catheter 110, and includes at least one RF electrode 114 having a plurality of electrically coupled segments 114A, a plurality of electrically isolated electrodes 114, or a combination thereof. The electrodes 114 are movably coupled to a support structure 290. More particularly, a resilient member 292 couples each electrode 114 or segment 114A to the support structure 290 such that each electrode 114 or segment 114A may be deflected independent of the other electrodes 114 or segments thereof. Thus, device 100 is analogous to a “bed of nails” with the electrode segments 114A being the nails. This device advantageously conforms to the anatomy of the tissue. The resilient member 292 may be formed of an electrically conductive material and may electronically couple the electrodes 114 to the conductors 230.

Support structure 290 is preferably formed of a resilient material to facilitate deployment through catheter 110. The resilient member 292 may be a spring or the like. Support structure 290 defines a plurality of receptacles 294 in which the electrodes 114 are movably retained.

The resilient member 292 may serve a dual purpose of retaining the electrode 114 within the receptacle 294 while permitting some relative movement between the electrode 114 and the receptacle 294.

Alternatively, the receptacle 294 may include a lip or flange (not illustrated) adapted to engage a corresponding lip (not illustrated) formed on the electrode 114 to retain the electrode 114.

According to one variation, any of the electrodes or energy delivery devices 114 contained in this disclosure may be non-coplanar. For example, an apparatus for delivering energy to tissue according to the present invention may include an elongate flexible shaft having a proximal end and a distal end. A flexible or resilient housing 112 may be provided on the distal end 110D of the flexible shaft, and one or more electrodes 114 may be mounted on the housing 112. If multiple electrodes 114 are provided, they may be electrically insulated from one another and/or may be spaced apart from one another. The electrodes 114 have a surface adapted to appose the tissue which has a shape conforming to the anatomy of a patient. According to one embodiment, the shape may define any non-planar shape e.g., a continuous curve or a step.

### Smart Electrode

Empirical evidence indicates that different tissue types have different electrical characteristics, including different impedance properties and electrocardiac conductivity. Moreover, there exist variations in electrical characteristics even within a given tissue type. These differences may be used to map the tissue in order to orient the device relative. In addition, the tissue electrical characteristics may be used as a feedback mechanism in controlling energy delivery. Tissue electrical characteristics may be used to optimize the amount of energy delivered to the tissue, the timing and rate in which it is delivered, and even the location to which it is delivered.

In the context of the PFO, the primum is generally thin tissue whereas the secundum is generally thicker tissue. Moreover, the septum primum responds differently than the secundum to RF energy. Notably, a given amount of RF energy results in a markedly smaller impedance decrease when delivered to the primum than the secundum, as well as a smaller temperature rise (gradient). This result is due to differences in the tissue characteristics and/or differences in tissue thickness.

In a device 100 according to the present invention it is possible to measure the impedance properties and/or the electrocardiac conductivity between two electrodes 114 or the impedance properties between an electrode 114 and the ground pad 234. The measured impedance properties and/or the electrocardiac conductivity will vary depending on the tissue's electrical characteristics as well as the distance between the two electrodes 114 (or electrode 114 and ground pad 234). By manner of illustration, the impedance properties and/or the electrocardiac conductivity may be measured in FIG. 4A between the first electrode 114 and any one of the second electrodes 114 by connecting either the first or second electrode 114 to the ground terminal of the generator (which action may be controlled by controller 228A). Alternatively, the impedance properties could be measured between an electrode and the ground terminal. The impedance properties and/or the electrocardiac conductivity may be measured in each of the RF electrode devices 100 described in this application. The use of additional electrodes 114 results in greater resolution, enabling the user to localize areas of varying impedance properties. Importantly, the impedance properties and/or the electrocardiac conductivity may be measured in real-time while energy is applied to the tissue and may used as a feedback mechanism by the controller 228A to control the amount of energy being applied to a given electrode 114.

In terms of tissue mapping, impedance properties and/or the electrocardiac conductivity may be used to distinguish between one or more tissue types. For example, the septum primum (primum) may have markedly different impedance and/or electrocardiac conductivity than the septum secundum (secundum). The septum primum is thinner than septum secundum, and has a lower absolute impedance. Further, the primum is composed of significantly less muscular tissue than secundum, and therefore the impedance will not decrease as dramatically in response to initial energy delivery. Due to the muscular tissue in the secundum, there is more electrocardiac activity in the secundum than the primum too. This information could be used for mapping because the PFO orientation and size (generally shaped like a frown) differs widely. Moreover, it is difficult to determine the orientation of the PFO frown using conventional echocardiography imaging devices. By measuring the tissue impedance properties and/or the electrocardiac conductivity using different electrodes the user may determine the orientation of the frown, and may utilize this information to orient the energy delivery device 114. Alternatively, the tissue impedance information could be used to selectively activate portions of the energy delivery device such that the energy delivery is optimized and specific to the location of the PFO.

According to one aspect of the invention, impedance properties and/or the electrocardiac conductivity may be used to orienting the energy delivery device. The method consists of providing a catheter device having a plurality of electrically independent electrodes, guiding the catheter device to a target location using at least one of a guide wire and imaging means, and measuring at least one of an impedance value and electrocardiac conductivity between a given pair of electrodes and adjusting the orientation and or position of the catheter device in accordance with the measured value (impedance/electrocardiac conductivity). Any conventional imaging means may be used to guide the catheter device 100 to the target location; however, ultrasound, transesophogeal echocardiogram (TEE), and transthoracic echocardiogram (TTE) are particularly useful. It is extremely difficult to determine the orientation of the electrodes 114 using conventional imaging hence the advantage of using impedance properties and/or the electrocardiac conductivity to orienting the energy delivery device.

The method for orienting the energy delivery device may for example be used to position the energy device on a PFO. More particularly, the method may be used to determine whether the electrode 114 is biased posterior or anterior of one of the primum and secundum. Similarly, the method may be used to determine whether the electrode 114 is biased superior or inferior of one of the primum and secundum. Moreover, by measuring the impedance properties and/or the electrocardiac conductivity it is possible to determine which electrodes 114 are positioned on the PFO tissues and selectively activate only electrodes that address the PFO.

The impedance properties and/or the electrocardiac conductivity may be used to determine the orientation of the PFO tunnel relative to the catheter axis.

The impedance properties and/or the electrocardiac conductivity may be used to determine at least one of the location, size, and orientation of one of the primum and the secundum.

A system for selectively delivering energy to tissue according to the present invention includes a multi-channel RF energy supply 228 including at least two independently adjustable channels. The device 100 may include any of the multi-electrode designs disclosed in this application. The electrically independent electrodes 114 are connected to the multi-channel RF energy supply 228, with at least one electrically independent electrode 114 connected to each of at least two channels such that energy applied to at least two electrodes 114 may be independently controlled. Controller 228A communicates with the multi-channel RF energy supply 228 and controls the delivery of energy to the electrodes 114A. The controller 228A measures the impedance between a given electrode 114 and the ground pad 234 or between a given pair of electrodes 114 and adjusts the amount and manner in which energy is delivered in accordance with the measured impedance.

As disclosed above, energy may be delivered in a monopolar, bipolar, or multipolar manner. Moreover, the energy may be delivered to each electrode 114 sequentially or simultaneously. According to some applications it may be advantageous to apply energy in a stepwise manner, e.g., first to one electrode 114 (or group of electrodes) then to two electrodes (or two groups of electrodes) simultaneously, then to three electrodes (or three groups of electrodes) simultaneously.

Each of the devices 100 disclosed herein may be provided with one or more thermocouples 240 for measuring the temperature of the tissue. According to one embodiment, plural thermocouples 240 are provided. The thermocouple 240 may communicate with the controller 228A which may terminate delivery of energy to one or more electrodes 114 in accordance with the measured temperature. The thermocouple(s) 240 may be mounted to the electrode 114, substrate 122, or distal housing 112.

According to a preferred embodiment, the device 100 includes plural thermocouples 240. For example, one thermocouple device 240 may be provided may be provided proximate each electrode 114. The controller 228A may utilize the temperature data from the thermocouples 240 as feedback to control the amount of energy being applied to the electrode(s) 114.

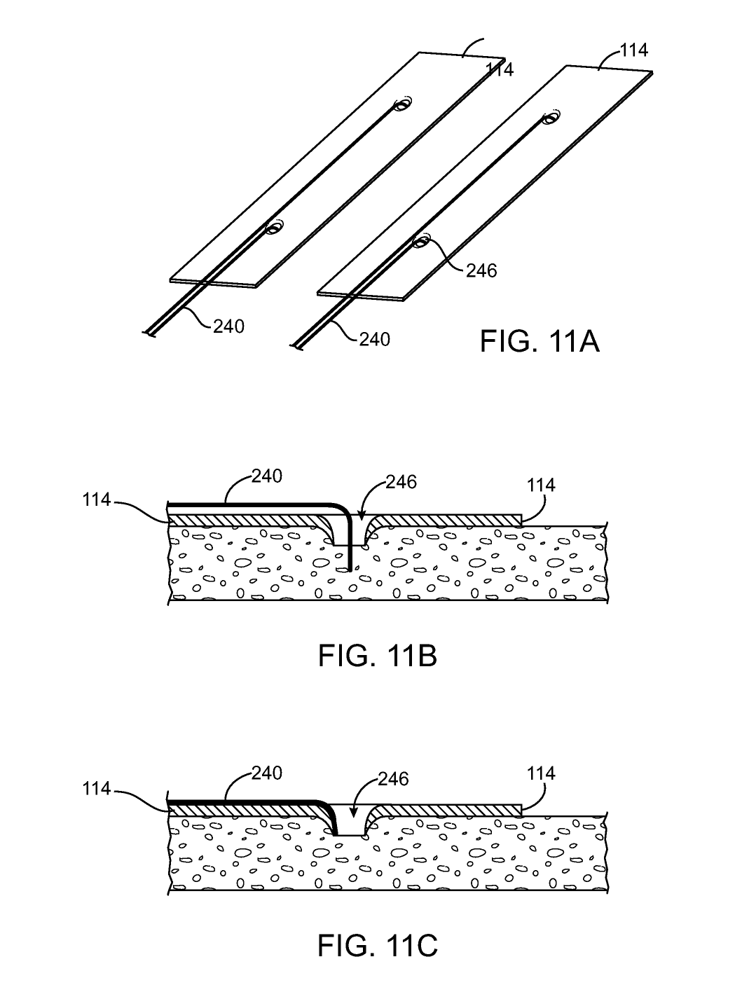

As shown in FIGS. 11A and 11B, the tissue apposition surface of the energy delivery device 114 may include a flange 242 configured to pierce or displace tissue, and a thermocouple 240 proximate the flange 242 for measuring the temperature of the displaced or pierced tissue. Moreover, the energy delivery device 114 may define an aperture 246 in fluid communication with the vacuum lumen for venting gases and the like. The flange 242 may partially surround the aperture 246, and the thermocouple 240 may be operably connected to the flange 242. In some embodiments, the flange 242 is frusto-conical and complete surrounds the aperture 246. In any event the precise shape of the flange 242 is not limited to any particular shape. Likewise, it is not necessary to include an aperture 246, and some embodiments simply include a flange for piercing or displacing tissue and a thermocouple for measuring the temperature of the pierced or displaced tissue.

Although the foregoing description is complete and accurate, it has described only exemplary embodiments of the invention. Various changes, additions, deletions and the like may be made to one or more embodiments of the invention without departing from the scope of the invention. Additionally, different elements of the invention could be combined to achieve any of the effects described above. Thus, the description above is provided for exemplary purposes only and should not be interpreted to limit the scope of the invention as set forth in the following claims.

What is claimed is:

1. An apparatus for delivering energy to tissue, comprising:
an elongate flexible shaft having a proximal end and a distal end;
a sheath disposed over at least a portion of the flexible shaft;
a housing provided on the distal end of the flexible shaft; and
a plurality of electrodes mounted on the housing, the electrodes having a tissue apposition surface having a non-coplanar shape that conforms to the anatomy of a patient.

2. The apparatus of claim 1, wherein the tissue apposition surface defines a continuous curve.

3. The apparatus of claim 1, wherein the tissue apposition surface defines a step.

4. The apparatus of claim 1, wherein the plurality of electrodes are oval shaped.

5. An apparatus for delivering energy to tissue, comprising:
an elongate flexible member having a proximal end and a distal end, the distal end of the elongate member being predisposed to assume a first predefined shape;
at least one electrode disposed on the elongate member proximate the distal end; and
a sheath disposed over at least a portion of the elongate member and adapted to house the elongate member in an undeployed state in which the elongate member generally conforms to the shape of the sheath.

6. The apparatus of claim 5, wherein the at least one electrode comprises at least one circumferential band disposed around the elongate member.

7. The apparatus of claim 5, wherein the predefined shape is generally one of an L-shape, a helix, a square, and a series of interlocking squares.

8. An apparatus for delivering energy to tissue, comprising:
an elongate flexible member having proximal end and a distal end;
an expandable and conformable member predisposed to assume a first predefined shape, said conformal member being operably connected to the distal end of the elongate member; and
a plurality of electrodes disposed on the expandable member wherein at least some of the electrodes are electrically independent from the remaining electrodes,
wherein the sheath is adapted to house the expandable member in a collapsed state prior to deployment.

9. The apparatus of claim 8, wherein the expandable member comprises a balloon.

10. The apparatus of claim 8, wherein the balloon comprises one of a continuously curved region and a stepped region.
